(19) United States
(12) Patent Application Publication
(10) Pub. No.: US 2005/0112566 A1
(43) Pub. Date: May 26, 2005

(54) Method and apparatus for the in-situ preparation of macromolecules via uniform glow discharge plasma

(76) Inventors: Kimberly Kelly-Wintenberg, Knoxville, TN (US); Daniel M. Sherman, Louisville, TN (US); Suzanne South, Seymour, TN (US)

Correspondence Address: PITTS AND BRITTIAN P C, P O BOX 51295, KNOXVILLE, TN, 37950-1295, US

(21) Appl. No.: 10/257,827

(22) PCT Filed: Oct. 10, 2001
(86) PCT No.: PCT/US01/31896

Related U.S. Application Data
(60) Provisional application No. 60/238,543, filed on Oct. 10, 2000.

(51) Int. Cl. B05D 3/00; C12Q 1/68; G01N 33/53

(52) U.S. Cl. 435/6; 427/2.11; 435/7.1

(57) ABSTRACT

The present invention is directed toward an apparatus for the in-situ preparation of macromolecules via uniform glow discharge plasma, and a method for using the apparatus. The method and apparatus are designed for preparing macromolecules from biological materials, including at least DNA, RNA, saccharides, lipids and proteins, in a manner which eliminates the need for biological solvents or chemicals, grinders, freezing, or detergents. The present invention is capable of operating at one atmosphere of pressure. The present method is a non-destructive, thus rendering the yielded macromolecules amenable for further modification or analysis via exposure to the glow discharge plasma sustained at substantially atmospheric pressure in air or modified gas environments. The device includes a spaced apart pair of metallic electrodes. At least one of the electrodes is covered with a high dielectric insulation material. A power supply is provided for energizing the electrodes. In the method, the biological material is placed on a substrate or suspended in solution and then placed within the device. The biological material is immersed in direct contact with the plasma or an active species generated by the plasma such that the exterior of the biological material is disrupted, yielding the macromolecules generally intact and available for analysis and/or modification.

### CROSS-REFERENCE TO RELATED APPLICATIONS

This application claims the benefit of U.S. Provisional Application No. 60/238,543 , filed Oct. 10, 2000.

### STATEMENT REGARDING FEDERALLY SPONSORED RESEARCH OR DEVELOPMENT

Not Applicable.

### BACKGROUND OF THE INVENTION

1. Field of Invention

This invention relates to an apparatus and a method for the in-situ preparation of macromolecules from biological materials. More specifically, the present invention relates to a method and apparatus for altering the membrane, capsid, or envelope of the biological material such that the contained macromolecules are accessible for further analysis and modification without necessarily requiring liquid suspension or lysing in the preparation of the biological material.

2. Description of the Related Art

In the field of the preparation of biological materials, there are several known methods for extracting biological material such as DNA, RNA, saccharides, lipids and proteins from a sample material. The most widely used method is a chemical method in which organic solvents are used to destabilize the cell membranes. In a mechanical or physical method, very small beads are used to beat against the cells to rip them open for extraction of the contents therein. In an ultrasonic method, ultrasonic waves are used to break open, or lyse, the cells.

Another known method uses a steady-state glow discharge plasma apparatus. Typical of the art are those methods or devices disclosed in the following U.S. Patents:

| U.S. Pat. No. | Inventor (s) | Issue Date |
|---|---|---|
| 4,954,320 | J. G. Birmingham et al. | Sep. 4, 1990 |
| 5,387,842 | J. R. Roth et al. | Feb. 7, 1995 |
| 5,403,453 | J. R. Roth et al. | Apr. 4, 1995 |
| 5,414,324 | J. R. Roth et al. | May 9, 1995 |
| 5,456,972 | J. R. Roth et al. | Oct. 10, 1995 |
| 5,669,583 | J. R. Roth | Sep. 23, 1997 |
| 5,938,854 | J. R. Roth | Aug. 17, 1999 |
| 5,989,824 | J. G. Birmingham et al. | Nov. 23, 1999 |
| 6,146,724 | J. R. Roth | Nov. 14, 2000 |

Those patents issued to either Roth or Roth et al., disclose the use of a steady-state glow discharge plasma apparatus in various applications. The apparatus is operated at one atmosphere of pressure. A pair of spaced apart insulated metallic electrodes are energized using low radio frequency with an rms potential of 1 to 20 KV at 1 to 100 kHz. Air or another gas such as helium or argon is passed between the electrodes. The electrodes are typically charged by a power supply and an impedance matching network adjusted to produce the most stable uniform glow discharge. The temperature of the electrodes is controlled to further assure the non-destructive aspects of the One Atmosphere Uniform Glow Discharge Plasma.

Sterilization of a wide variety of microorganisms has been accomplished using this type of uniform glow discharge plasma. The sterilization is caused by interrupting the integrity of the biological material. This interruption is caused by reactive oxygen species which damages the biological material via toxicity, disruption, and leaking of the macromolecules. The ability of the plasma to prepare macromolecules for analysis and modification is the same process as plasma sterilization except the exposure times are shorter.

In the discipline of physics, the term “plasma” describes a partially ionized gas composed of ions, electrons and neutral species. This state of matter may be produced by the action of either very high temperatures, strong electric or radio frequency (R.F.) electromagnetic fields. High temperature or “hot” plasmas are represented by celestial light bodies, nuclear explosions and electric arcs. Glow discharge plasmas are produced by free electrons which are energized by an imposed direct current (DC) or R.F. electric fields and then collide with neutral molecules. These neutral molecule collisions transfer energy to the molecules and form a variety of active species including metastables, atomic species, free radicals and ions. These active species are chemically active and/or physically modify the surface of materials and may therefore serve as the basis of new chemical compounds and property modifications of existing compounds.

Low power plasmas known as corona discharges have been widely used in the surface treatment of thermally sensitive materials such as paper, wool and synthetic polymers such as polyethylene, polypropylene, polyolefin, nylon and poly(ethylene terephthalate). Because of their relatively low energy content, corona discharge plasmas can alter the properties of a material surface though the filamentary nature of the corona may damage the surface.

Glow discharge plasmas represent another type of low power density plasma useful for non-destructive material surface modification. However, glow discharge plasmas have heretofore been generated typically in low pressure or partial vacuum environments below 10 torr, necessitating batch processing and the use of expensive vacuum systems. Some glow discharges can be generated at atmospheric pressure in a manner such that there is a high degree of spatial uniformity if an ion trapping mechanism is employed.

In the '824 patent issued to Birmingham et al., bacterial cells or spores are collected and concentrated to form a specimen that is lysed using an ionized fluid to facilitate identification of the sample by tests performed on the DNA or RNA contained therein. An impact collector is used to separate the sample from an air sample that is drawn through. The sample is then exposed to an ionizing discharge to rupture the surface membrane of the cells.

### BRIEF SUMMARY OF THE INVENTION

The present invention is directed toward an apparatus for the in-situ preparation of macromolecules via uniform glow discharge plasma, and a method for using the apparatus. The method and apparatus are designed for preparing macromolecules from biological materials, including at least DNA, RNA, saccharides, lipids and proteins, in a faster and more economical manner than is typically possible by eliminating the need for biological solvents or chemicals, grinders, freezing, or detergents to remove the macromolecules from the biological material. More specifically, the present invention is directed to the in situ preparation of surfaces or liquids holding biological material using the active species from a glow discharge plasma which is capable of operating at one atmosphere of pressure. The present method is non-destructive, thus rendering the yielded macromolecules amenable for further modification or analysis via exposure to the glow discharge plasma sustained at substantially atmospheric pressure in air or modified gas environments.

The device includes a spaced apart pair of electrodes. The electrodes are fabricated from either a solid metal plate, a porous metal, or a metallic mesh material. At least one of the electrodes is covered with a high dielectric insulation material. In the embodiment wherein one of the electrodes is fabricated from a porous surface or metallic mesh, that electrode is not covered with the dielectric material.

A low impedance, high voltage, radio frequency (RF) power amplifier is provided for energizing the electrodes. The power amplifier, or power supply has independently variable voltage and frequency capacities over the respective ranges of 1 to at least 20 KV and 1 to 100 KHz.

Surrounding the plate assembly is an environmental isolation barrier such as a structural enclosure suitable for maintaining a controlled gas atmosphere in the projected plan volume between the electrodes Gas pressure within the isolation barrier is substantially ambient, thereby obviating or reducing the need for gas tight seals.

In the method of the present invention for preparing macromolecules derived from a biological material, the biological material is placed on a substrate. When the biological material is in solution, it may be attached to the substrate by placing the solution on the substrate and then drying it, thus leaving a residue on the substrate. When the biological material is in a growth, it is attached to the substrate in a conventional manner. In either situation, direct exposure of either the substrate with the attached biological material or suspension of the biological material in an analysis solution, the biological material is immersed in direct contact with the plasma. Alternatively, indirect exposure of the substrate with the attached biological material or suspension of the biological material in the analysis solution is such that the biological material is bathed in the convected active species generated by the plasma.

After placing the biological material on the substrate or in suspension, the substrate or analysis solution is enclosed in a protective barrier for being exposed to a plasma or plasma active species at a selected pressure. The substrate is positioned on the dielectric coating of an electrode, or, in the embodiment wherein one of the electrodes remains uncoated, directly on the uncoated electrode. It will be understood that the electrode or dielectric material could be used as the substrate. In the situation where the biological material is suspended in an analysis solution, the electrode or dielectric material is useful in some applications to contain the analysis solution. Still further, in the embodiment wherein the electrodes are fabricated from a porous metal or metallic mesh, an electrode may also serve as a lid to the analysis solution container.

The substrate or analysis solution is placed in the device in which a steady-state radio frequency (RF) uniform glow discharge plasma is generated at a pressure of from 10 torr to 20 bar. The uniform glow discharge plasma is generated at a preferred pressure of one (1) atmosphere. The RF waves are regulated at a frequency from about 100 Hz to about 30 kHz with an electric field of from about 1 to about 20 kV/cm. The electric field is produced for trapping ions produced by the plasma, without trapping electrons produced by the plasma. The biological material is then exposed either directly or indirectly to the generated plasma, interrupting the integrity of the membranes, capsid, or envelope such that the interior macromolecular materials leak out and are accessible. The period of exposure is determined by the composition of the biological material or the composition of the analysis solution, and more specifically, is limited such that only the exterior of the biological material is torn, yielding the macromolecules generally intact. After the macromolecular material has been obtained from within its outer covering, analysis and/or modification of the exposed macromolecules is performed.

### BRIEF DESCRIPTION OF THE SEVERAL VIEWS OF THE DRAWINGS

The above mentioned features of the invention will become more clearly understood from the following detailed description of the invention read together with the drawings in which:

FIG. 1 is a perspective illustration of a device constructed in accordance with several features of the present invention;

FIG. 2 is a front elevation view, in section taken along lines 2-2 of the device of FIG. 1;

FIG. 3 is a top plan view, in section along lines 3-3 in FIG. 2 of the device of FIG. 1;

FIG. 4 is a schematic illustration of one embodiment of a power supply incorporated in the device of FIG. 1;

FIG. 5 illustrates an alternate power supply similar to that of FIG. 4, and further including a grounded center tap transformer;

FIG. 6 illustrates a further alternate power supply;

FIG. 7 illustrates an alternate embodiment of the power supply of FIG. 6, further including impedance matching circuitry and a transformer;

FIG. 8 illustrates the amplification results of Pseudomonas aeruginosa and Staphylococcus aureus after exposure to atmospheric plasma for 15 and 30 seconds using standard protocols;

FIG. 9 illustrates the amplification results of Pseudomonas aeruginosa and Staphylococcus aureus after exposure to standard protocols versus atmospheric plasma protocols;

FIG. 10 illustrates the Density Gradient Gel Electrophoresis (DGGE) analysis of Pseudomonas aeruginosa and Staphylococcus aureus after exposure to standard protocols versus atmospheric plasma protocols;

FIG. 11 a gel image of amplification products of dilute Bacillus niger spores exposed to atmospheric plasma for 135 seconds;

FIG. 12 illustrates the amplification results of Saccharomyces cerevisiae after exposure to atmospheric plasma for 30 and 60 seconds and placed in amplification tubes with 50 μl DNA grade water, 0.25 M Tris buffer, or 0.01 M TE;

FIG. 13 illustrates the amplification products of Aspergillus niger after exposure to atmospheric plasma for 30 and 45 seconds;

FIG. 14 illustrates Aspergillus niger exposed to plasma at atmospheric pressure for 90 seconds; and

FIG. 15 illustrates the amplification products of bacteriophage after exposure to atmospheric plasma for 60, 120, 135, 150 and 165 seconds and placed into amplification tubes with water, 50 μl of 0.25 M Tris and 0.01 M TE.

### DETAILED DESCRIPTION OF THE INVENTION

An apparatus for the in-situ preparation of macromolecules via uniform glow discharge plasma incorporating various features of the present invention is illustrated generally at 10 in the figures. A method for using the apparatus 10 is described. The method and apparatus 10 are designed for preparing macromolecules from biological materials, including at least DNA, RNA, saccharides, lipids and proteins, in a faster and more economical manner than is typically possible by eliminating the need for biological solvents or chemicals, grinders, freezing, or detergents to remove the macromolecules from the biological material. More specifically, the present invention is directed to the in situ preparation of surfaces or liquids holding biological material using the active species from a glow discharge plasma which is capable of operating at one atmosphere of pressure. The present method is a non-destructive, in situ method of preparing macromolecules including at least DNA, RNA, proteins, lipids, and saccharides derived from biological materials such as bacteria, spores, fungi, and viruses. The method renders such macromolecules amenable for further modification or analysis via exposure to the glow discharge plasma sustained at substantially atmospheric pressure in air or modified gas environments.

Extraction of the macromolecules occurs by the energetic etching, destruction or perforation of the exterior portion of the biological material that is in contact with atmospheric electrical plasma or reactive oxidated species created by the plasma. Biological component extraction is accomplished by one of several methods including corona, One Atmosphere Uniform Glow Discharge Plasma (OAUGDP), and microwave plasma discharges. Other methods of non-thermal atmospheric discharge may be employed as well. By exposing a material containing the biological agent, the plasma discharge disrupts the continuity of the membranes, capsid, or envelope such that the interior macromolecular materials leak out and are accessible for analyses. Such protocol is performed very rapidly, i.e., a few seconds to a few minutes. It is performed on dry or semi-dry surfaces, materials or containers, or on liquid surfaces. There are no by-products being formed, which eliminates time consuming chemical extraction of the biological macromolecules from the organisms.

The present invention incorporates the device originally disclosed by Roth et al., in U.S. Pat. No. 5,387,842, and as modified in subsequent patents issued to Roth et al. collectively, and to Roth individually. Disclosure of those patents as listed above is incorporated herein by reference.

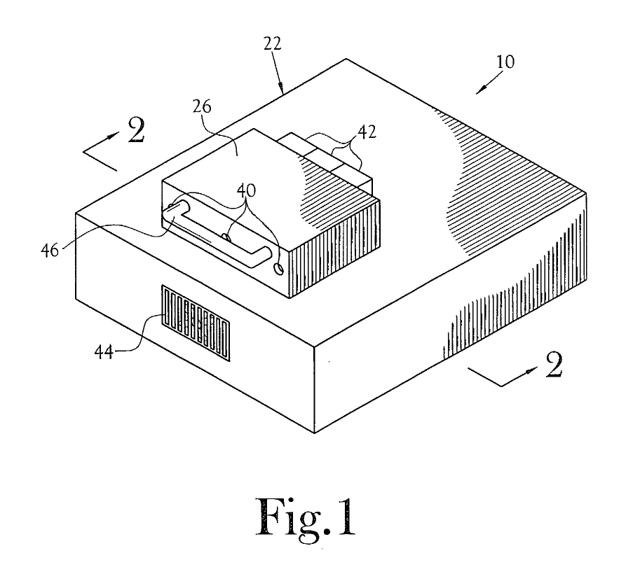

The device 10 of the present invention is illustrated by FIGS. 1-3. FIG. 1 illustrates in perspective view the device 10. A housing 22 with a lid 26 hingeably attached to the top thereof. Discussed in further detail below, inlets 40 are defined on one end of the lid 26 to allow the inlet of air drawn by fans 42 disposed on an opposite side of the lid 26 Further, a fan 44 is provided on the front of the housing 22 for inlet of air to the housing 22. A handle 46 is provided for lifting the lid 26.

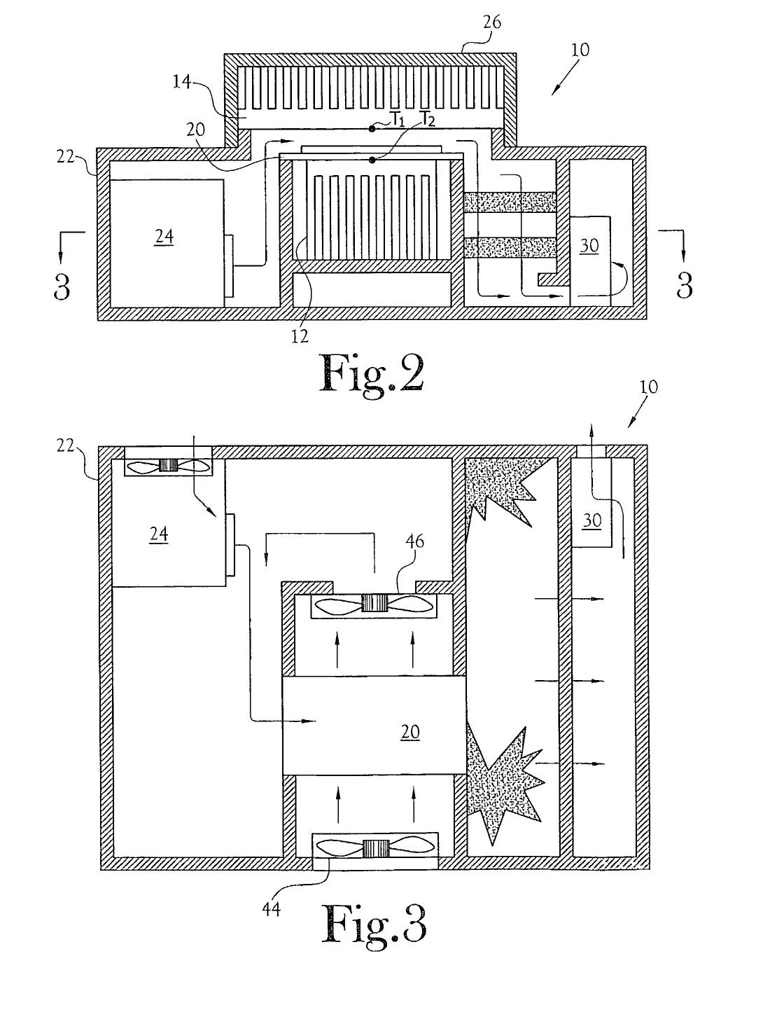

As illustrated in FIG. 2, electrodes 12,14 are provided in a spaced apart disposition. In one embodiment, the electrodes 12,14 are fabricated from a metallic plate. In an alternate embodiment, one of the electrodes 12,14 is fabricated from a porous surface or metallic mesh. The upper electrode 12 is mounted in the lid 26 such that as the lid 26 is opened, the electrodes 12,14 are separated for the placement or removal of biological material applied to a substrate 34 or suspended in an analysis solution. At least one of the electrodes 12,14 is covered with a high dielectric insulation material 20. In the embodiment wherein one of the electrodes 12,14 is fabricated from a porous surface or metallic mesh, that electrode 12,14 is not covered with the dielectric material 20, primarily for economical reasons, and the other of the electrodes 12,14 is covered with the high dielectric insulation material 20.

A low impedance, high voltage, radio frequency (RF) power supply 24 is provided for energizing the electrodes 12,14 at contacts T₁ and T₂, respectively. The power supply 24, of the preferred embodiment has independently variable voltage and frequency capacities over the respective ranges of 1 to at least 20 KV and 1 to 100 KHz.

As illustrated in FIG. 3, a fan 44 cooperates with a fan 46 for drawing ambient air under the lower electrode 14 to draw heat from the electrode 14. Air drawn through the fans 44,46 is combined with air introduced into the housing 22 via the power supply 24. This air is then passed between the electrodes 12,14, through the ozone destruction material 28 and then evacuated from the housing 22 via the blower 30. In the preferred embodiment, the electrodes 12 are maintained at a temperature below 65° Celsius. Although ambient air is disclosed as being used, a gas selected from ambient air, oxygen, nitrous oxide, carbon tetraflouride, carbon dioxide, nitrogen, a noble gas such as helium, neon, and argon, and mixtures thereof may be used.

Gas pressure within the housing 22 is substantially ambient, thereby obviating or reducing the need for gas tight seals. Normally, it is sufficient to maintain a low flow rate of the modified atmosphere gas through the housing 22 that is sufficient to equal the leakage rate. Since the pressure within the housing 22 is essentially the same as that outside the housing 22, there is substantially no pressure differential to create leakage.

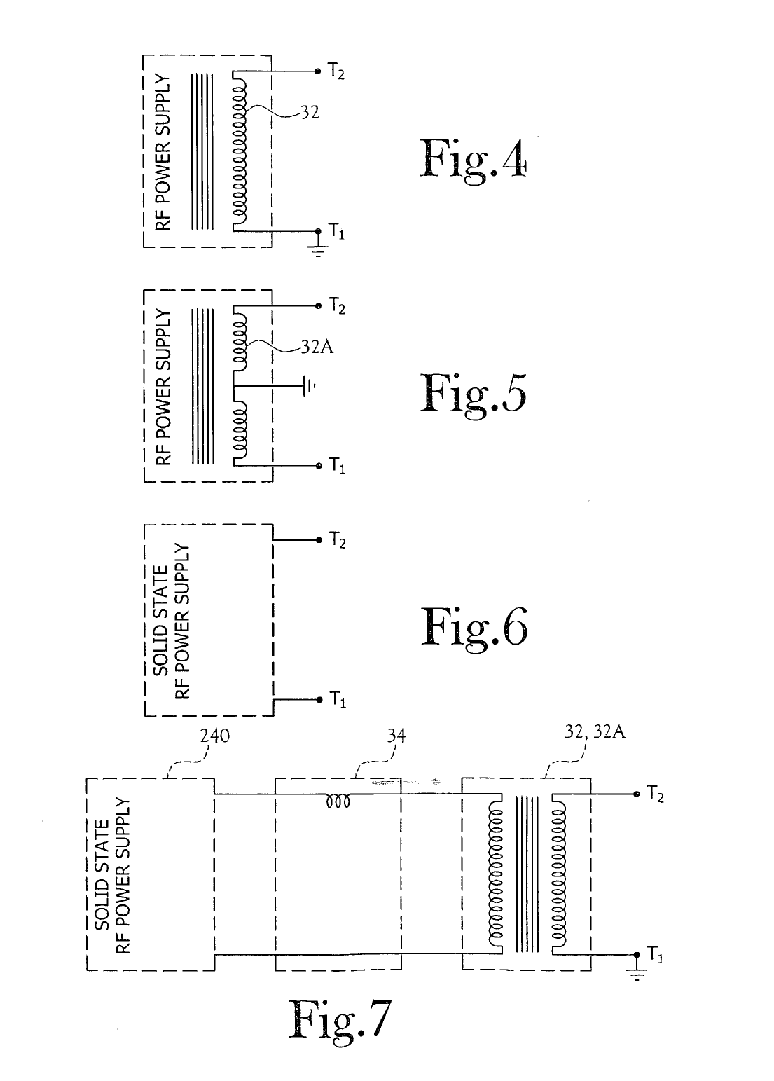

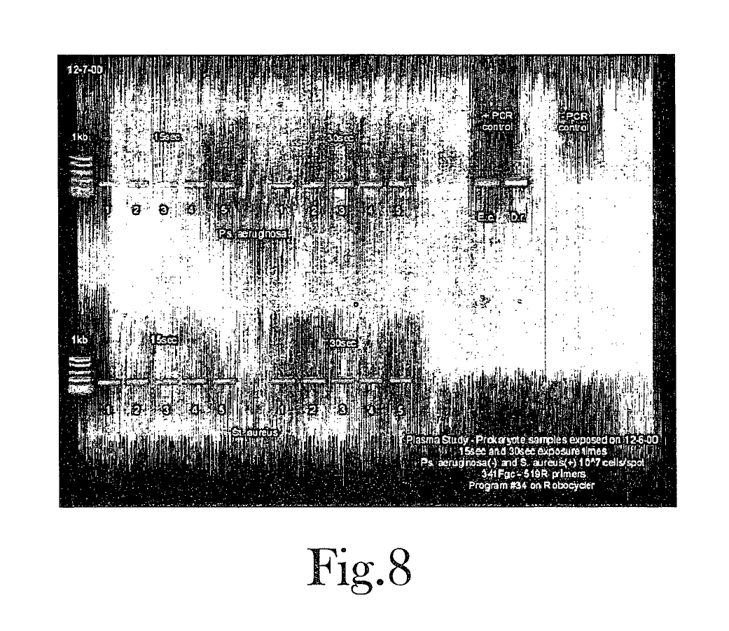

FIGS. 4, 5, 6 and 7 illustrate various embodiments of the power supply 24. In FIG. 4, the power supply 24A includes a transformer 32 having contacts T₁ and T₂, the voltages of which are 180 degrees out of phase, but at only half the maximum potential. FIG. 5 illustrates a power supply 24B similar to that of FIG. 4, and further includes a grounded center tap transformer 32A. FIG. 6 illustrates a solid state power supply 24C. As in the previous embodiments, this embodiment may or may not include a grounded center tap. FIG. 7 illustrates a solid state power supply 24D similar to the power supply 24C of FIG. 6, further including impedance matching circuitry 34 and a transformer 32,32A. As designated, the transformer 32,32A may be either of those illustrated in FIGS. 4 and 5.

In the method of the present invention for preparing macromolecules derived from a biological material, the biological material is placed on a substrate 34 or suspended in an analysis solution. The substrate 34 is fabricated from any of a variety of materials including, but not limited to, metal, ceramic, glass, plastic, polymer, paper, film, filter, and rubber material. While a planar configuration is illustrated, the substrate 34 may alternatively define a simple curvature. When the biological material is in solution, it may be attached to the substrate 34 by placing the solution on the substrate 34 and then drying it, thus leaving a residue on the substrate 34. The solution is dried using either an evaporative or a heat-based method or is filtered onto a substrate. When the biological material is in a growth, it is applied to the substrate 34 in a conventional manner. In either situation, direct exposure of either the substrate 34 with the applied biological material or suspension of the biological material in an analysis solution, the biological material is immersed in direct contact with the plasma. Alternatively, indirect exposure of the substrate 34 with the applied biological material or suspension of the biological material in the analysis solution is such that the biological material is bathed in the convected active species generated by the plasma.

After placing the biological material on the substrate or in suspension, the substrate or analysis solution is enclosed in a protective barrier 26 for being exposed to a plasma or plasma active species at a selected pressure. The substrate is illustrated as being positioned on the dielectric coating of an electrode, or, in the embodiment wherein one of the electrodes 12 remains uncoated, directly on one of the electrodes 12. It will be understood that the electrode or dielectric material could be used as the substrate. In the situation where the biological material is suspended in an analysis solution, the electrode 12 or dielectric material 20 is useful in some applications to contain the analysis solution. Still further, in the embodiment wherein the electrodes 12 are fabricated from a porous metal or metallic mesh, an electrode 12 may also serve as a lid to the analysis solution container.

In the preferred embodiment, the substrate 34 or analysis solution is placed in the device 10 of the present invention in which a steady-state radio frequency (RF) uniform glow discharge plasma is generated at a pressure of from 10 torr to 20 bar. In the preferred method of the present invention, the uniform glow discharge plasma is generated at a pressure of one (1) atmosphere. The RF waves are regulated at a frequency from about 100 Hz to about 30 kHz with an electric field of from about 1 to about 20 kV/cm. The electric field is produced for trapping ions produced by the plasma, without trapping electrons produced by the plasma. The biological material is then exposed either directly or indirectly to the generated plasma, interrupting the integrity of the membranes, capsid, or envelope such that the interior macromolecular materials leak out and are accessible. The period of exposure is determined by the composition of the biological material or the composition of the analysis solution, and more specifically, is limited such that only the exterior of the biological material is disrupted, yielding the macromolecules generally intact. After the macromolecular material has been obtained from within its outer covering, analysis and/or modification of the exposed macromolecules is performed.

The device and method of the present invention have been utilized in a number of applications. Following are results of several evaluations.

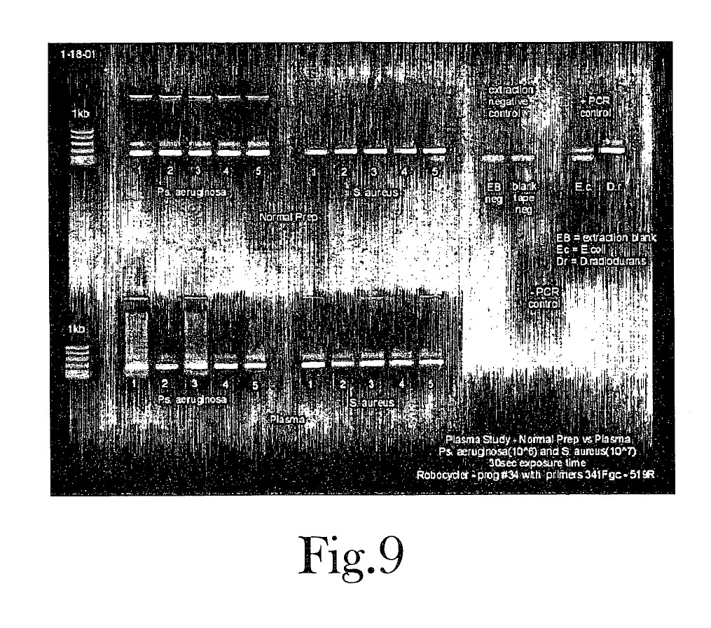

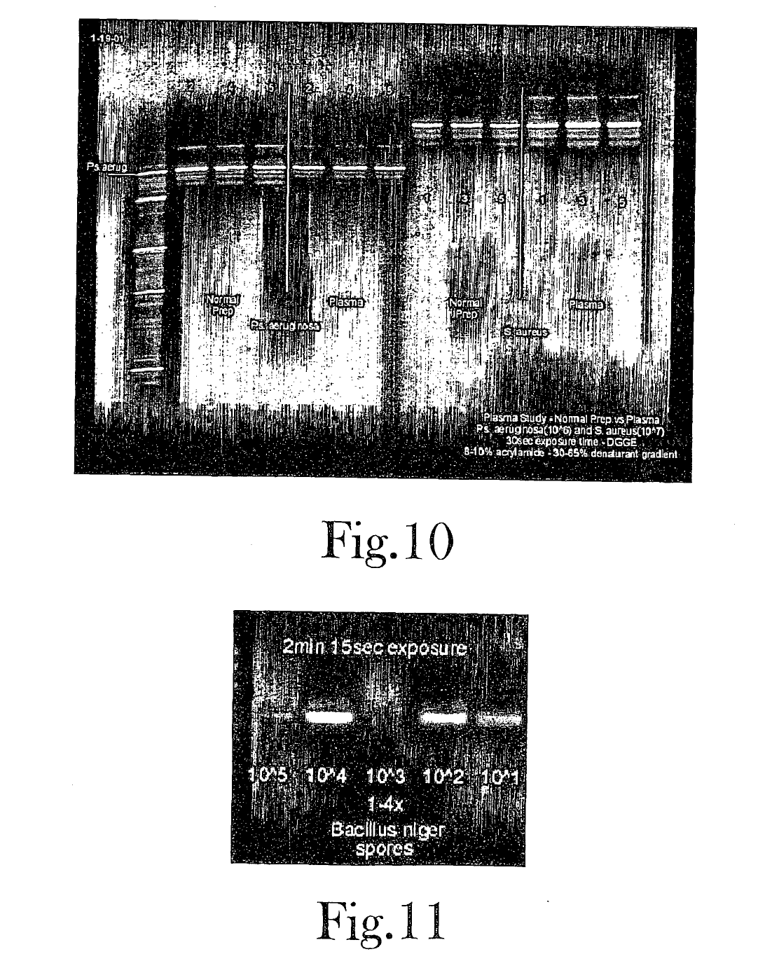

Bacterial samples of Pseidonmotias aeruginosa and Siaphylococcus aureus were inoculated onto sterile strips of electromagnetic tape at working concentrations of 10⁷ and 10⁶ cells/spot, respectively. The samples were placed in the device 10 and were exposed to plasma at atmospheric pressure for 15 and 30 seconds. The samples were then placed into amplification tubes containing 1 μL of 0.01 M TE (10 mM Tris, 100 mM EDTA). See FIG. 8. Control samples of Pseudomonas aeruginosa and Staphylococcus aureis were prepared by placing 10⁷ cells in an extraction tube and adding 425 μL phosphate buffer (pH 8.0), 175 μL of CRSR Lysis buffer, and 0.5 grams of sterile, DNA-free glass beads. The cell suspension was then placed into a Savant FP 120 Fast Prep system and bead beat for 45 seconds at maximum speed. DNA was then extracted from the suspension using a standard Phenol/Chloroform protocol. See FIG. 9. Amplification of the 16S rDNA region was performed using primers 519R (ATTACCGCGGCTGCTGG) and 341Fgc (CGCCCGCCGCGCGCGGCGGGCCCGGGGCGGGGGCACGGGGGGCCTACGGGAGGCAGCAG). Amplification was performed using a Stratogene Robocycler, using DNA from Escherichia coli and D. radiodurans as amplification controls. The samples were processed in a thermocycler at 94° C. for 2 minutes (1 cycle); 94° C. for 20 seconds, 56° C. for 45 seconds, 72° C. for 45 seconds (34 cycles); and 72° C. for 5 minutes (1 cycle). The DNA products were then analyzed using Density Gradient Gel Electrophoresis (DGGE). See FIG. 10. The ladder in lane 1 was spiked with the 16s rDNA amplification product prepared by the standard protocol.

DGGE was performed with a Bio-Rad Protean 11 system. PCR samples were applied directly onto 8% wt/vol polyacrylamide gels in 0.5×TAE (20 mM Tris acetate (pH 7.4), 10 mM sodium acetate, 0.5 mM Na₂EDTA) with gradients which were formed with 8% wt/vol acrylamide stock solutions and which contained 0 and 100% denaturant (7 M urea) and 40% formamide deionized with AG501-X8 mixed bed resin (Bio-Rad). Electrophoresis was performed as a constant voltage of 200V and a temperature of 60° C. After electrophoresis, the gels were incubated for 15 minutes in molecular-grade water containing ethidium bromide, rinsed, and imaged.

Samples of dilute Bacillus niger spores were spotted onto sterile strips of electromagnetic tape at working concentrations of 10⁴, 10² and 10 cells/spot. The samples were exposed to plasma at atmospheric pressure for 135 seconds. The spots were then cut from the tape and placed in amplification tubes containing 50 μL of 0.25 M Tris. Amplification of the 168 rDNA region was performed using primers 519R and 341fcg. The samples were processed in the thermocycler in a fashion similar to the bacterial samples above. A gel image of the amplification products is illustrated in FIG. 11. Lanes 1, 2 and 4 indicate concentrations of 10, 10², and 10⁴ cells/spot. Lane 6 indicates a 1 Kbase ladder. Lanes 3 and 5 were empty.

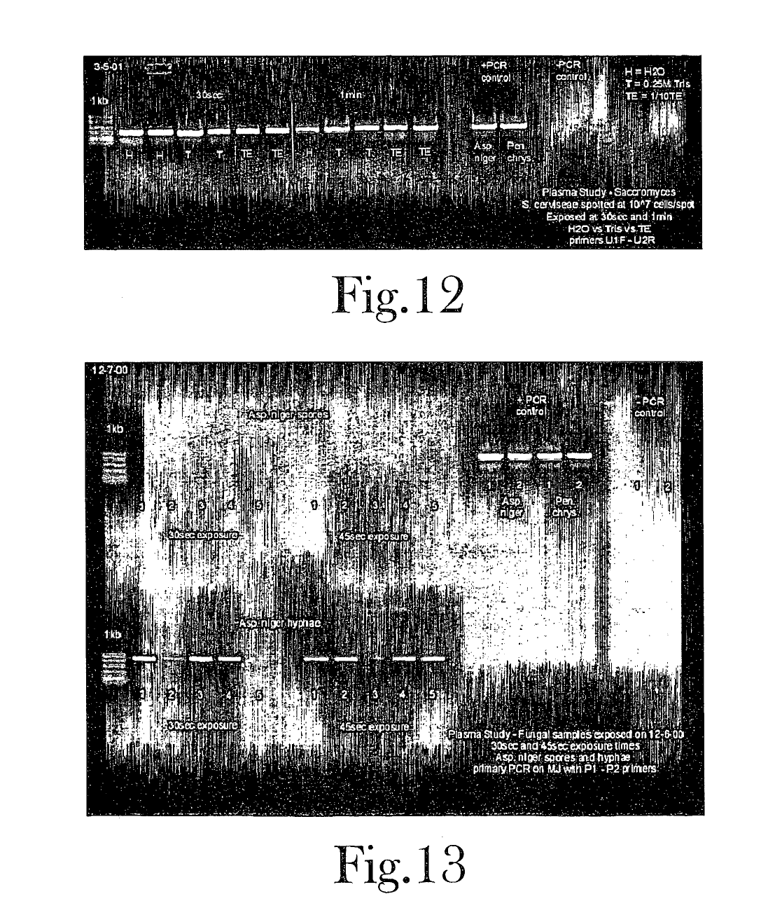

Yeast samples of Saccdaromyces cerevisiae were applied to sterile electromagnetic tape at a concentration of 10⁷ cells/spot and exposed to plasma at atmospheric pressure for 30 seconds or 60 seconds. Sections of tape were then placed in amplification tubes with 50 μL DNA grade water, 0.25 M Tris buffer, or 0.01 M TE. Control samples of Saccharomnyces cerevisiae were prepared as described for the bacterial controls described above. A nested PCR amplification of the 28S rDNA region was performed using primers P1F (ATCAATAAGCGGAGGAAAAG) and P2R (CTCTGGCTTCACCCTATTC) for the primary reaction of U1F (GTGAAATTGTTGAAAGGGAA) and U2R (GACTCCTTGGTCCGTGTT) for the secondary reaction. The P1-P2 samples were processed in a thermocycler at 94° C. for 2 minutes (1 cycle); 94° C. for 15 seconds, 40° C. for 30 seconds, 72° C. for 1 minute (34 cycles); and 72° C. for 5 minutes (1 cycle). The U1-U2 samples were processed in a thermocycler at 94° C. for 2 minutes (1 cycle); 94° C. for 15 seconds, 50° C. for 30 seconds, 72° C. for 1 minute (34 cycles); and 72° C. for 5 minutes (1 cycle). DNA isolated from Aspergillus niger and Penicillium chrysoperium by the standard method was used as an amplification control. FIG. 12 illustrates the images derived from the exposures times of 30 seconds and 60 seconds for the yeast samples suspended in the solutions of water (H), Tris buffer (T), and Tris-EDTA (TE).

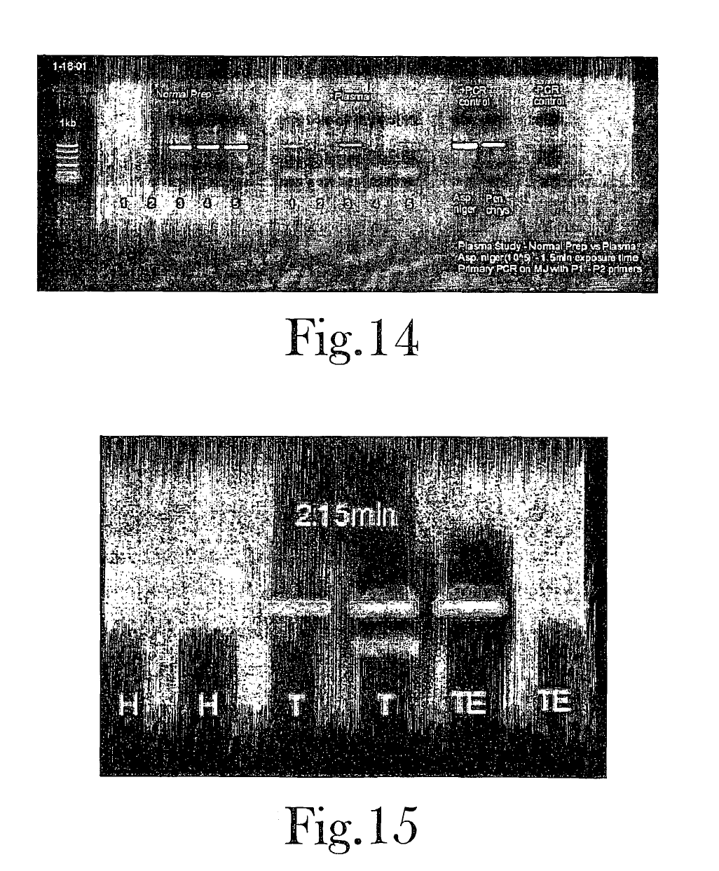

Fungal samples of Aspergillus niger were placed on sterile electromagnetic tape and exposed to plasma at atmospheric pressure for 30, 45 or 90 seconds. Sections of the tape with the hyphae were then cut and placed in amplification tubes containing 0.25 M Tris. PCR amplification of the 28S rDNA region was performed as described above using primers P1F-P2R and U1F-U2R. Aspergillus niger spores were suspended in sterile, DNA-free water, and AODC counts were performed. Spores were spotted onto the magnetic tape at 10⁵ spores/spot and exposed to plasma at atmospheric pressure for 90 seconds. Sections of the tape spotted with the spores were placed in amplification tubes and processed as described above. Control samples of Aspergillus niger and Penicillium chrysoperium were prepared as described for the bacterial control samples. As illustrated in FIG. 13, samples exposed for 30 and 45 seconds provided amplification products similar to that of the standard protocol. By comparison, hyphae exposed to plasma at atmospheric pressure for 90 seconds provided amplification product that was not as robust as the standard protocol, as illustrated in FIG. 14. Spores of Aspergillus niger exposed for 60 seconds provided an amplification product with a marginal signal.

Viral samples of bacteriophage (T2 phage) was spotted onto sterile electromagnetic tape at 10⁸ PFU/spot. Samples were exposed to plasma at atmospheric pressure for times of 60, 120, 135, 150 or 165 seconds. The sample spots were then cut from the tape and placed into amplification tubes with 50 μL of 0.25 M Tris. PCR amplification was performed using primers T2F (CAAGCTGCTGATAACGAT) and T2Rev (CTAGTAAGGTCATTCGCTGC) designed using the conserved regions of gene 36. The samples were processed in a thermocycler at 94° C. for 2 minutes (1 cycle); 94° C. for 20 seconds, 56° C. for 45 seconds, 72° C. for 45 seconds (34 cycles); and 72° C. for 5 minutes (1 cycle). As depicted in FIG. 15, T2 phage exposed to the plasma for 135 seconds provided the most robust amplification product and was equivalent to the standard protocol. Longer exposures to the plasma gave inconsistent results. T2 phage samples amplified with 0.25 M Tris as the starting buffer provided the most consistent amplification product. Lane 1 contains the 1 kb DNA ladder. Lanes 2 and 3 represent the samples suspended in water (H), lanes 4 and 5 represent those sample s suspended in 0.25M Tris, and lanes 6-7 represent those samples suspended in 0.01M TE buffer.

From the foregoing description, it will be recognized by those skilled in the art that a method and apparatus for the in-situ preparation of macromolecules via uniform glow discharge plasma offering advantages over the prior art has been provided. Specifically, the method and apparatus are designed for preparing macromolecules from biological materials in a faster and more economical manner than is typically possible by eliminating the need for biological solvents or chemicals, grinders, freezing, or detergents. The method and apparatus are designed for the in situ preparation of surfaces or liquids holding biological material using the active species from a glow discharge plasma which is capable of operating at one atmosphere of pressure. The present method is non-destructive, thus rendering such macromolecules amenable for further modification or analysis via exposure to the glow discharge plasma sustained at substantially atmospheric pressure in air or modified gas environments.

While a preferred embodiment has been shown and described, it will be understood that it is not intended to limit the disclosure, but rather it is intended to cover all modifications and alternate methods falling within the spirit and the scope of the invention as defined in the appended claims.

1. A method for preparing macromolecules derived from a biological material, said method comprising the steps of:
a) preparing the biological material using a selected medium;
b) generating a steady-state radio frequency (RF) uniform glow discharge plasma at a pressure of from 10 torr to 20 bar;
c) exposing said biological material to said discharge plasma for a set period of time;
d) interrupting the integrity of an outer covering of the biological material such that the interior macromolecular materials leak out and are accessible; and
e) collecting the macromolecules for at least one of performing analysis of the macromolecules and modification of the macromolecules.

2. The method of claim 1 wherein said macromolecules include at least one of DNA, RNA, saccharides, lipids and proteins from the biological material.

3. The method of claim 1 wherein said step of preparing the biological material using a selected medium includes the step of applying the biological material to a substrate.

4. The method of claim 3 wherein step of applying the biological material to a substrate is accomplished by evaporation to apply the biological material to said substrate.

5. The method of claim 3 wherein said step of applying the biological material to a substrate is accomplished by drying using heat to dry the biological material onto said substrate.

6. The method of claim 3 wherein said substrate is fabricated from a material selected from the group consisting of metals, ceramics, glasses, plastics, polymers, papers, webs, filters, films and rubbers.

7. The method of claim 3 wherein said substrate is permeable and wherein said step of applying the biological material to a substrate includes the step of filtering the biological material onto said permeable substrate.

8. The method of claim 1 wherein said step of preparing the biological material using a selected medium includes the step of suspending the biological material in an analysis solution in a container.

9. The method of claim 1 wherein said step of generating a steady-state radio frequency (RF) uniform glow discharge plasma is accomplished at about one atmospheric pressure.

10. The method of claim 1 wherein said step of generating a steady-state radio frequency (RF) uniform glow discharge plasma is accomplished at a frequency of from 100 Hz to 30 kHz.

11. The method of claim 10 wherein said step of generating a steady-state radio frequency (RF) uniform glow discharge plasma is accomplished such that said uniform glow discharge plasma defines an electric field of from 1 to 20 kV/cm.

12. The method of claim 11 wherein said step of generating a steady-state radio frequency (RF) uniform glow discharge plasma is accomplished such that said electric field traps ions without trapping electrons, wherein the ions and the electrons are produced by the plasma.

13. The method of claim 1 wherein said step of exposing said biological material to said discharge plasma is accomplished by direct exposure of the biological material much that the biological material is immersed in direct contact with the plasma.

14. The method of claim 1 wherein said step of exposing said biological material to said discharge plasma is accomplished by indirect exposure of the biological material to the plasma such that the biological material is immersed in direct contact with a convected active species/gas generated by the plasma.

15. The method of claim 1, after said step of preparing the biological material using a selected medium, and before said step of generating a steady-state radio frequency (RF) uniform glow discharge plasma, further comprising the step of enclosing the biological material in a protective barrier for being exposed to either of a plasma and plasma active species or a selected pressure.

16. The method of claim 1 wherein the plasma is generated from at least one of the group consisting of ambient air, oxygen, nitrous oxide, carbon tetraflouride, carbon dioxide, nitrogen and, noble gases, including helium, neon, and argon.

17. An apparatus for preparing macromolecules from a biological material, the biological material having an outer covering in which is stored the macromolecules, said apparatus comprising:
a first electrode and second electrode, said first and second electrodes being spaced apart in parallel fashion, the biological material being disposed between said first and second electrodes;
a power supply in electrical communication between said first and second electrodes for generating a steady state, radio frequency (RF), uniform glow discharge plasma between said first and second electrodes at a pressure of from about 10 torr to about 20 barr; and
an enclosure for receiving at least said first and second electrodes, said plasma and said biological material.

18. The apparatus of claim 17 wherein the biological material is applied to a substrate, said substrate being receivable between said first and second electrodes.

19. The apparatus of claim 17 wherein the biological material is suspended in an analysis solution in a container receivable between said first and second electrodes.

20. The apparatus of claim 17 configured to place the biological material in direct exposure of the generated plasma for a set period of time to interrupt the integrity of the outer covering of the biological material to allow leakage of the macromolecules.

21. The apparatus of claim 17 configured to place the biological material in indirect exposure of the generated plasma and in direct exposure of a convected active species/gas generated by the plasma for a set period of time to interrupt the integrity of the outer covering of the biological material to allow leakage of the macromolecules.

22. The apparatus of claim 17 wherein at least one of said first and second electrodes is covered with a solid dielectric material.

23. The apparatus of claim 17 further comprising a cooling device for removing heat generated by each of said first and second electrodes.

24. The apparatus of claim 17 wherein said first and second electrodes are electrically connected to said power supply which operates to produce an electric field for trapping ions generated by said plasma without trapping electrons generated by said plasma such that spatial uniformity of said plasma is optimized.
